(19) United States
(12) Patent Application Publication
(10) Pub. No.: US 2015/0145690 A1
(43) Pub. Date: May 28, 2015

(54) HEALTH IMPROVEMENT SYSTEM AND METHOD AND NON-TRANSITORY COMPUTER-READABLE STORAGE MEDIUM THEREOF

(71) Applicant: INSTITUTE FOR INFORMATION INDUSTRY, TAIPEI (TW)

(72) Inventors: Cheng-Yuan HO, Tapei City (TW); I-Hsuan CHIU, New Taipei City (TW)

(73) Assignee: INSTITUTE FOR INFORMATION INDUSTRY, TAIPEI (TW)

(21) Appl. No.: 14/184,680

(22) Filed: Feb. 19, 2014

(30) Foreign Application Priority Data
Nov. 25, 2013 (TW) 102142883

(51) Int. Cl. A61B 5/00

(52) U.S. Cl. CPC A61B 5/0024
USPC 340/870.07

(57) ABSTRACT

A health improvement system includes at least one sensor module, a computing module, an interaction module and a database module. The sensor module is configured for detecting at least one data selected from the group consisting of user behaviors, user activities, user physical data, user psychological data, and a combination thereof, so as to generate sensor data. The computing module is configured for computing the sensor data, for generating an evaluation result to evaluate an effect for at least one user's health, for converting the sensor data and the evaluation result to at least one internal parameter, and for changing a health condition representation according to the internal parameter. The interaction module is configured for outputting the health condition representation. The database module is configured for storing the user input parameters, the sensor data, the evaluation result, the internal parameters and the health condition representation.

### RELATED APPLICATIONS

This application claims priority to Taiwan Application Serial Number 102142883, filed Nov. 25, 2013, the entirety of which is herein incorporated by reference.

### BACKGROUND

1. Technical Field

The present disclosure relates to a health improvement system. More particularly, the present disclosure relates to a health improvement system and method, which convert users' behaviors, the users' activities and the resulting effects on the users' health to the changes of virtual characters related to or chosen by the users.

2. Description of Related Art

Due to the busy lives, it is not easy for people to have enough rest, to drink enough water or to exercise regularly. Therefore, there are more and more health improvement devices or systems, which remind people to keep good living habits. For example, a computer system is configured for recording the daily sleeping time or a pedometer is configured for counting the daily walking steps and for calculating the consumed calories.

However, the incentive effects of the above current health improvement devices or systems are not satisfactory for the users due to some reasons including that the input of user behavior data, user activity data, and so on is not convenient and the output is restricted in simple records or analyses.

### SUMMARY

In one aspect, the present disclosure is related to a health improvement system. The health improvement system includes at least one sensor module, a computing module, an interaction module and a database module. The sensor module is configured for detecting at least one data selected from the group consisting of user behaviors, user activities, user physical data, user psychological data, and a combination thereof, so as to generate sensor data. The computing module is configured for computing the sensor data, for generating an evaluation result to evaluate an effect for at least one user's health, for converting the sensor data and the evaluation result to at least one internal parameter, and for changing a health condition representation according to the internal parameter. The interaction module is configured for receiving at least one user input parameter and for outputting the health condition representation. The database module is configured for storing the user input parameters, the sensor data, the evaluation result, the internal parameters and the health condition representation.

In another aspect, the present disclosure is related to a health improvement method. The health improvement method includes the following steps: receiving at least one user input parameter; detecting the at least one data selected from the group consisting of user behaviors, user activities, user physical data, user psychological data, and a combination thereof, so as to generate sensor data; computing the sensor data and generating an evaluation result to evaluate an effect for at least one user's health; converting the sensor data and the evaluation result to at least one internal parameter; changing a health condition representation according to the internal parameter; storing the user input parameters, the sensor data, the evaluation result, the internal parameters and the health condition representation; and outputting the health condition representation.

According to another embodiment of this disclosure, a non-transitory computer-readable storage medium storing a computer program for executing the steps of the aforementioned health improvement method is provided. Steps of the method are as disclosed above.

These and other features, aspects, and advantages of the present disclosure will become better understood with reference to the following description and appended claims.

It is to be understood that both the foregoing general description and the following detailed description are by examples, and are intended to provide further explanation of the disclosure as claimed.

### BRIEF DESCRIPTION OF THE DRAWINGS

The disclosure can be more fully understood by reading the following detailed description of the embodiment, with reference made to the accompanying drawings as follows:

FIG. 1 is a schematic diagram of a health improvement system in accordance with one embodiment of the present disclosure;

FIG. 2 is a schematic diagram of a health improvement system in accordance with one embodiment of the present disclosure;

FIG. 3 is a flow chart of a health improvement method in accordance with one embodiment of the present disclosure;

FIG. 4 is a flow chart of a health improvement method in accordance with one embodiment of the present disclosure;

FIG. 5 is a flow chart of a health improvement method in accordance with one embodiment of the present disclosure;

FIG. 6 is a flow chart of a health improvement method in accordance with one embodiment of the present disclosure;

FIG. 7 is a flow chart of a health improvement method in accordance with one embodiment of the present disclosure;

FIG. 8 is a flow chart of a health improvement method in accordance with one embodiment of the present disclosure.

### DETAILED DESCRIPTION

Reference will now be made in detail to the present embodiments of the disclosure, examples of which are illustrated in the accompanying drawings. Wherever possible, the same reference numbers are used in the drawings and the description to refer to the same or like parts.

Unless otherwise defined, all terms (including technical and scientific terms) used herein have the same meaning as commonly understood by one of ordinary skill in the art to which example embodiments belong. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

In the following description and claims, the terms “coupled” and “connected”, along with their derivatives, may be used. In particular embodiments, “connected” and “coupled” may be used to indicate that two or more elements are in direct physical or electrical contact with each other, or may also mean that two or more elements may be in indirect contact with each other. “Coupled” and “connected” may still be used to indicate that two or more elements cooperate or interact with each other.

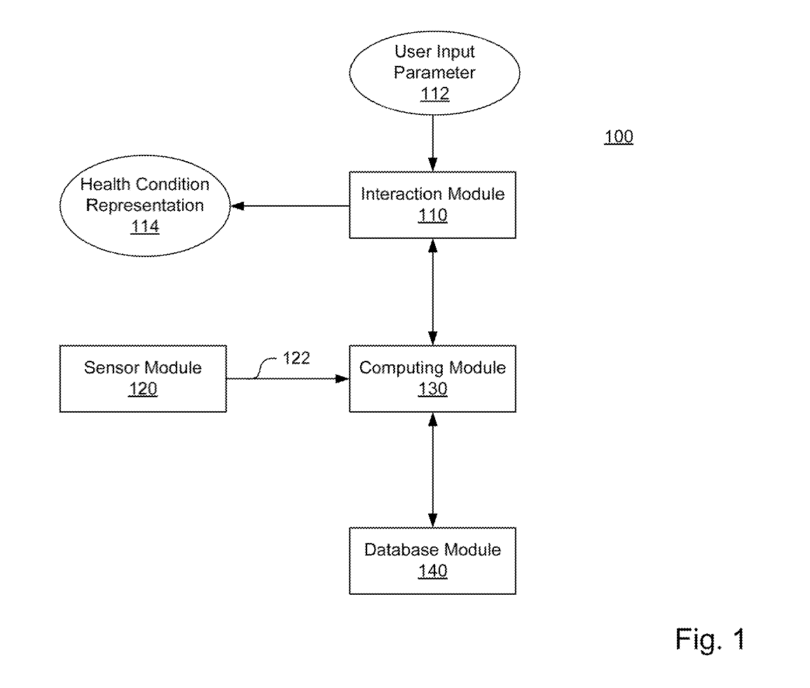

FIG. 1 is a schematic diagram of a health improvement system 100 in accordance with one embodiment of the present disclosure.

The health improvement system 100 includes an interaction module 110, at least one sensor module 120, a computing module 130, and a database module 140.

The interaction module 110 is configured for receiving at least one user input parameter 112 and for outputting a health condition representation 114.

In an embodiment of the present disclosure, the interaction module 110 could be implemented in, but not limited to, a smart phone, a personal computer, a laptop, a smart glasses or a smart watch. In another embodiment of the present disclosure, the user input parameters 112 include user age data, user gender data, user height data, user weight data, a parameter for choosing virtual characters, a parameter for adjusting difficulty level, a system order, a game order, or a combination thereof.

In one another embodiment of the present disclosure, the health condition representation 114 is an appearance icon of a virtual character. For example, the health condition representation 114 could be the appearance icon of a human figure, a cartoon character, an idol figure, a doll, an animal, a planet, a mineral, and so on showed on the display panel of electronic devices, such as desktop computers, laptops, smart phones, tablets, or portable devices.

The sensor module 120 is configured for detecting the at least one data selected from the group consisting of user behaviors, user activities, user physical data, user psychological data, and a combination thereof, so as to generate sensor data 122.

In an example, the sensor module 120 includes a pedometer and a GPS device. The pedometer is configured for counting the walking steps, the running steps or a combination thereof of the user, and for generating the sensor data 122. The GPS device is configured for recording the geographic location, the distance and the range of user activities, and for generating the sensor data 122. In another example, the sensor module 120 includes an electronic coaster. The electronic coaster is configured for measuring the weight of a glass, for calculating the amount of water drank by the user accordingly, and for generating the sensor data 122. In one another example, the sensor module 120 includes an electronic smart kettle. The electronic smart kettle is configured for measuring the variation of the water level and of the water weight, for calculating the amount of water drank by the user accordingly, and for generating the sensor data 122.

In one another embodiment of the present disclosure, the computing module 130 is configured for computing the sensor data 122, for generating an evaluation result to evaluate an effect for the at least one user's health, for converting the sensor data 122 and the evaluation result to at least one internal parameter, and for changing the health condition representation 114 according to the internal parameter.

In an embodiment of the present disclosure, the health improvement system 100 includes memory and one or more processors. The computing module 130 is stored in the memory and configured for execution by the one or more processors. In another embodiment of the present disclosure, the health improvement system 100 further includes a storage medium. The computing module 130 is stored in the storage medium when the health improvement system 100 shuts down. The computing module 130 is accessed from the storage medium, loaded into the memory and configured for execution by the one or more processors when the health improvement system 100 boots up.

In an example, a user would like to encourage himself to drink water self-motivatedly by utilizing the health improvement system 100. The user chose a dandelion icon as the health condition representation 114, and the sensor module 120 is implemented in an electronic coaster. The electronic coaster is configured for measuring the amount of water drank by the user as the sensor data 122. The computing module 130 is configured for calculating the daily amount of water drank by the user, and for generating an evaluation result to evaluate the effect for the user's health corresponding to the calculated daily amount of water drank by the user. The abovementioned evaluation result includes: the endangerment for the user's health resulted from the daily amount of water drank by the user being less than the recommended minimum daily amount of drinking water for the user, the endangerment for the user's health resulted from the daily amount of water drank by the user being far more than the recommended maximum daily amount of drinking water for the user, and the benefit due to the daily amount of water drank by the user reaching the recommended daily amount of drinking water for the user. The abovementioned recommended daily amount of drinking water for the user could be set by the use or be calculated by the computing module 130 according to the user age data, the user gender data, the user height data and the user weight data in the user input parameters 112. The computing module 130 is further configured for converting the abovementioned evaluation result of endangerment for the user's health to an internal parameter ‘endangerment’, for converting the abovementioned evaluation result of benefit for the user's health to an internal parameter ‘benefit’, and for converting the abovementioned daily amount of water drank by the user to an internal parameter ‘amount of drinking water’, in which ‘amount of drinking water’ is a numeric value with unit: liter. In this embodiment, when ‘endangerment’ occurs in an internal parameter, the computing module 130 changes the dandelion icon (the health condition representation 114) to a dehydrated and faded dandelion icon; when ‘benefit’ occurs in an internal parameter, the computing module 130 changes the dandelion icon (the health condition representation 114) to a vigorous, a blossomy or a fructifying dandelion icon.

In an embodiment of the present disclosure, the computing module 130 is further configured for searching and determining whether user data exists, wherein the user data is generated from the user input parameters 112 or the sensor data 122. If the user data exists, the computing module 130 is configured for loading the user data and the health condition representation 114. If the user data doesn't exist, the computing module 130 is configured for generating the user data and the health condition representation 114 according to the user input parameters 112.

In an example, the user would like to encourage himself to do exercise self-motivatedly by utilizing the health improvement system 100. After the health improvement system 100 is activated, the computing module 130 searches for the user data and determines whether the user data exists. If the user data exists, the computing module 130 is configured for loading the user data and a first animal icon (e.g., a Shiba Inu dog icon), which was chosen previously by the user as the health condition representation 114. If the user data doesn't exist (possible reasons include that the user has not used the health improvement system 100 before, or the user has deleted the user data from the health improvement system 100), the computing module 130 is configured for generating the user data and a second animal icon (e.g., a Golden Retriever dog icon) as the health condition representation 114 according to the user input parameters 112.

In an embodiment of the present disclosure, the computing module 130 is further configured for adjusting a difficulty level according to the user input parameters 112. For example, in the abovementioned example of encouraging the user to drink water self-motivatedly, if a 18-year-old male user is 165 cm high and 65 kg weigh, and the user input parameters 112 include a difficulty level parameter ‘easy’, the computing module 130 sets the recommended daily amount of drinking water for the user as 2 liters; and if the user input parameters 112 include the difficulty level parameter ‘hard’, the computing module 130 sets the recommended daily amount of drinking water for the user as 2.5 liters.

In an embodiment of the present disclosure, the computing module 130 is further configured for determining whether user activity data meets a passing-game condition, a warning condition or a special event. When the user activity data meets the passing-game condition, the warning condition or the special event, the interaction module 110 is further configured for informing the user.

In an example, the user would like to encourage himself to do exercise self-motivatedly by utilizing the health improvement system 100. The user chooses a Labrador Retriever dog icon as the health condition representation 114. In the health improvement system 100, the passing-game condition is set as “at least 30 minutes of exercise everyday for a week”, the warning condition is set as “less than 30 minutes of exercise for two consecutive days”, and the special event is set as “exercising for 60 minutes in a day”. When the computing module 130 determines the user activity data meets the abovementioned passing-game condition, the interaction module 110 outputs a message “You have exercised self-motivatedly for a week! Congratulations for pass the game!” to inform the user that he/she has reached the abovementioned passing-game condition. When the computing module 130 determines the user activity data meets the abovementioned warning condition, the interaction module 110 outputs a message “Warning! You have less than 30 minutes of exercise for two consecutive days!” When the computing module 130 determines the user activity data meets the abovementioned special event, the interaction module 110 outputs a message “You have exercised for 60 minutes today! Please keep going!”

In another embodiment of the present disclosure, when the user activity data meets the special event, the computing module 130 is further configured for generating award data, and for determining whether the user executes the award data according to the user input parameters 112. When the user executes the award data, the computing module 130 is further configured for changing the health condition representation 114.

For example, in the abovementioned example, when the computing module 130 determines the user activity data meets the special event (exercising for 60 minutes in a day), the computing module 130 generates the award data corresponding to the difficulty level (for example, icon data of a scarf for the Labrador Retriever). The interaction module 110 then shows the scarf icon on the display panel of the abovementioned electronic devices and asks if the user would like to use the scarf icon (i.e., the Labrador Retriever wears the scarf). If the user answers ‘yes’, the computing module 130 changes the health condition representation 114 (e.g., an icon of the Labrador Retriever dog with the scarf), and the interaction module 110 shows the health condition representation 114 as the Labrador Retriever dog with the scarf icon accordingly on the display panel of the abovementioned electronic devices.

The database module 140 is configured for storing the user input parameters 112, the sensor data 122, the evaluation result, the internal parameters and the health condition representation 114. For example, the database module 140 could be a flash memory of a mobile electronic device or a hard disk of a computer.

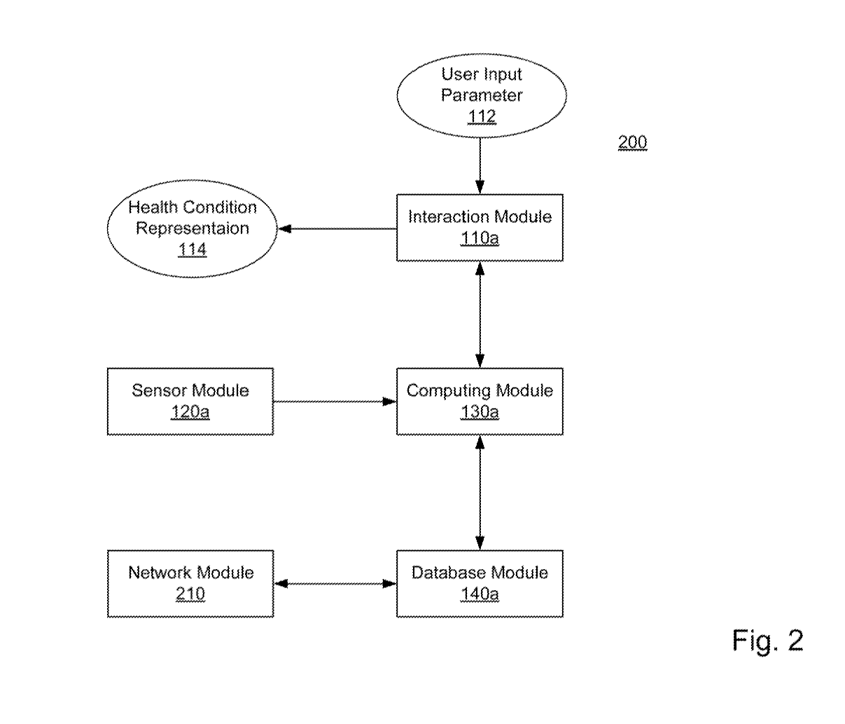

FIG. 2 is a health improvement system 200 in accordance with one embodiment of the present disclosure.

The health improvement system 200 includes an interaction module 110a, at least one sensor module 120a, a computing module 130a, a database module 140a and a network module 210.

The interaction module 110a, the sensor module 120a, the computing module 130a and the database module 140a could be the interaction module 110, the sensor module 120, the computing module 130 and the database module 140 illustrated in FIG. 1, respectively. Their functions and operations are similar and hence are not described again herein.

The network module 210 is configured for exchanging information generated by the computing module 130a or stored in the database module 140a with at least one external device. In an example, the network module 210 could be a wireless network module, a transmitter or an antenna of a mobile electronic device or a network adapter of a computer. In one embodiment of the present disclosure, the abovementioned external device includes another health improvement system 200, a social network, a server, or a combination thereof. In another embodiment of the present disclosure, the abovementioned information includes the health condition representation 114, achievement information, competition information or ranking information.

In an example, the user uploads the health condition representation 114 (for example, a Labrador Retriever dog icon) to Facebook and shares it with his/her friends by utilizing the network module 210. In another example, the user uploads the achievement information to Facebook or other health improvement systems 200 to show his/her friends the achievement he/she has done by utilizing the network module 210. In one another example, the user sends the competition information to other health improvement systems 200 and races with other users for the amount of daily exercise by utilizing the network module 210. In one another embodiment of the present disclosure, one hundred health improvement systems 200 are connected to a server or connected to each other by utilizing the network module 210 and having a competition for the amount of daily exercise at the server, and the server sends the ranking information to the network module 210 of the abovementioned one hundred health improvement systems 200.

From the above embodiments, by detecting the at least one data selected from the group consisting of user behaviors, user activities, user physical data, user psychological data, and a combination thereof, and converting the at least one data selected from the group consisting of user behaviors, user activities, user physical data, user psychological data, and a combination thereof to the changes of virtual characters correspondingly, the users could understand the effect for their health resulting from their behaviors, activities, physical data, psychological data, or a combination thereof. In this way, the users are encouraged to keep good habits and their health conditions are improved.

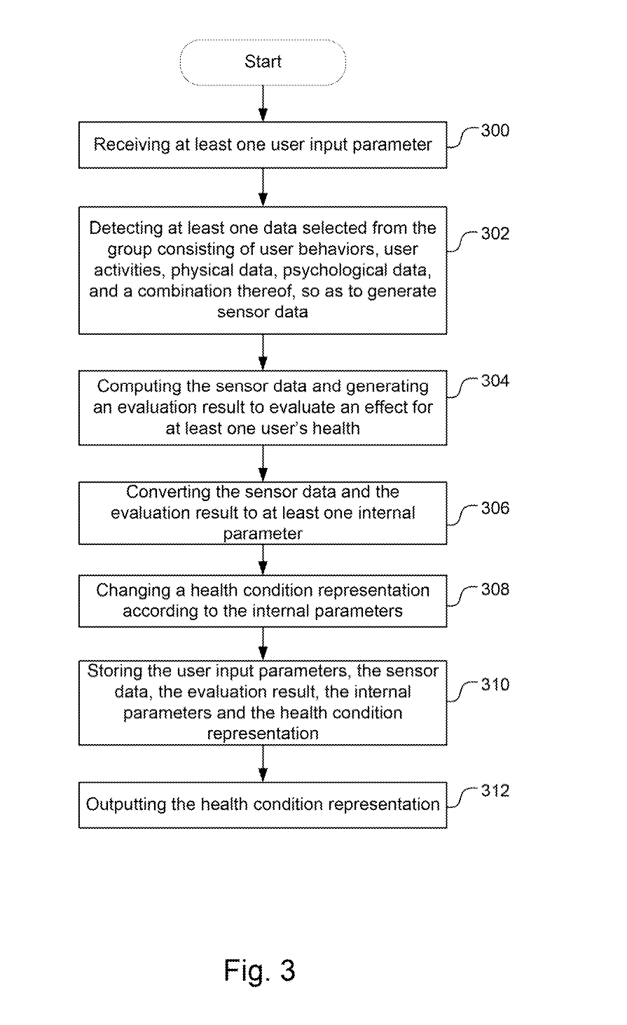

FIG. 3 is a flow chart of a health improvement method in accordance with one embodiment of the present disclosure. The health improvement method may take the form of a computer program product stored on a non-transitory computer-readable storage medium having computer-readable instructions embodied in the medium. Any suitable storage medium (such as non-transitory medium) may be used including, but not limited to, non-volatile memory such as read only memory (ROM), programmable read only memory (PROM), erasable programmable read only memory (EPROM), and electrically erasable programmable read only memory (EEPROM) devices; volatile memory such as static random access memory (SRAM), dynamic random access memory (DRAM), and double data rate random access memory (DDR-RAM); optical storage devices such as compact disc read only memories (CD-ROMs) and digital versatile disc read only memories (DVD-ROMs); and magnetic storage devices such as hard disk drives (HDD) and floppy disk drives.

The health improvement method could be applied in the health improvement system 100 illustrated in FIG. 1 and is not limited herein. For convenience and clarity, the following descriptions about the health improvement method are illustrated based on the health improvement system 100 illustrated in FIG. 1.

In step 300, the interaction module 110 is configured for receiving the at least one user input parameter 112 (for example, the user input parameters 112 include a system start order). In step 302, the sensor module 120 detects the at least one data selected from the group consisting of user behaviors, user activities, user physical data, user psychological data, and a combination thereof, so as to generate the sensor data 122. In step 304, the computing module 130 computes the sensor data 122 and generates an evaluation result to evaluate an effect for the at least one user's health. In step 306, the computing module 130 converts the sensor data 122 and the evaluation result to the at least one internal parameter. In step 308, the computing module 130 changes the health condition representation 114 according to the internal parameter. In step 310, the database module 140 stores the user input parameters 112, the sensor data 122, the evaluation result, the internal parameters and the health condition representation 114. In step 312, the interaction module 110 outputs the health condition representation 114.

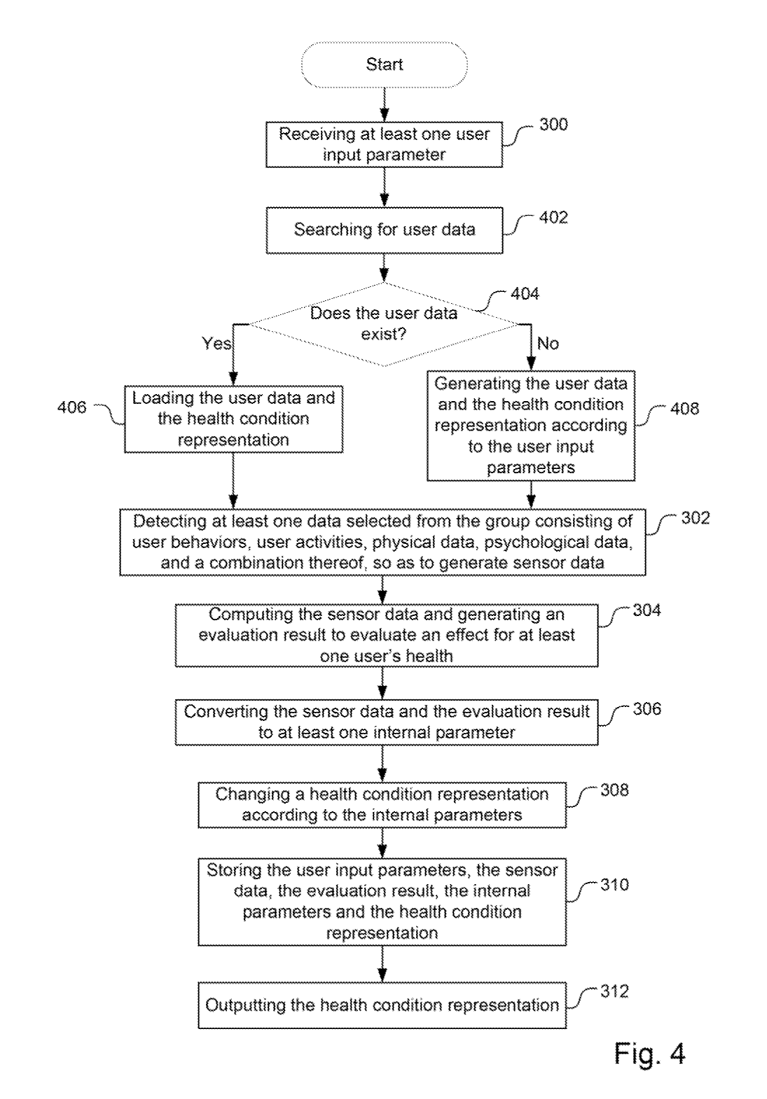

FIG. 4 is a flow chart of a health improvement method in accordance with another embodiment of the present disclosure.

The health improvement method could be applied in the health improvement system 100 illustrated in FIG. 1 and is not limited herein. For convenience and clarity, the following descriptions about the health improvement method are illustrated based on the health improvement system 100 illustrated in FIG. 1.

Compared with the health improvement method illustrated in FIG. 3, the health improvement method illustrated in FIG. 4 further includes steps 402, 404, 406 and 408. In step 402, the computing module 130 searches for the user data. In step 404, the computing module 130 determines whether the user data exists. If the user data exists, in step 406, the computing module 130 loads the user data and the health condition representation 114. If the user data does not exist, in step 408, the computing module 130 generates the user data and the health condition representation 114 according to the user input parameters 112.

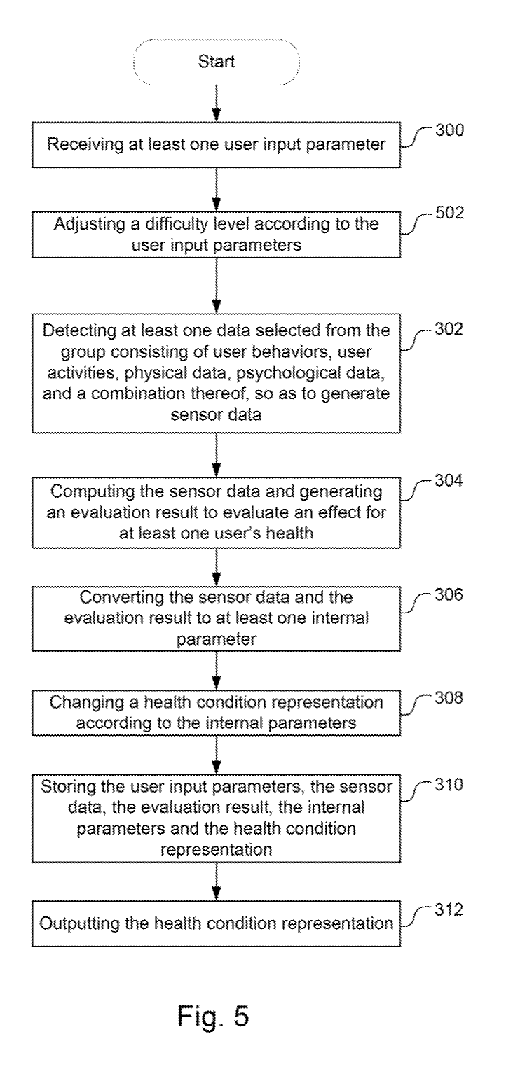

FIG. 5 is a flow chart of a health improvement method in accordance with one another embodiment of the present disclosure.

The health improvement method could be applied in the health improvement system 100 illustrated in FIG. 1 and is not limited herein. For convenience and clarity, the following descriptions about the health improvement method are illustrated based on the health improvement system 100 illustrated in FIG. 1.

Compared with the health improvement method illustrated in FIG. 3, the health improvement method illustrated in FIG. 5 further includes step 502. In step 502, the computing module 130 adjusts the difficulty level according to the user input parameters 112.

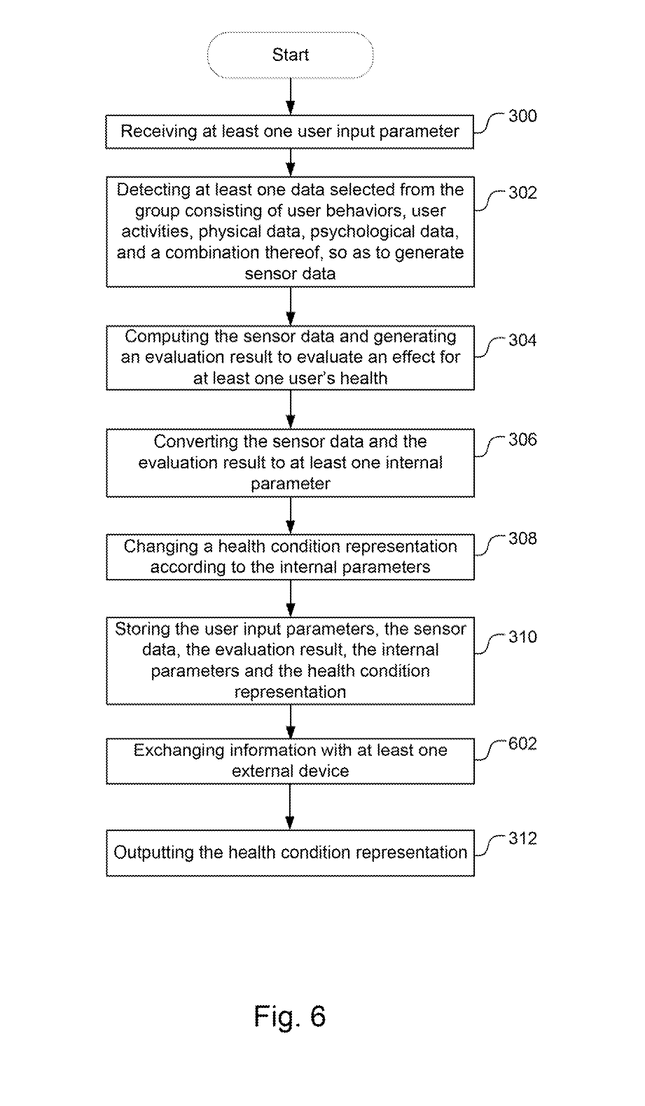

FIG. 6 is a flow chart of a health improvement method in accordance with one another embodiment of the present disclosure.

The health improvement method could be applied in the health improvement system 200 illustrated in FIG. 2 and is not limited herein. For convenience and clarity, the following descriptions about the health improvement method are illustrated based on the health improvement system 200 illustrated in FIG. 2.

Compared with the health improvement method illustrated in FIG. 3, the health improvement method illustrated in FIG. 6 further includes step 602. In step 602, the network module 210 exchanges the information generated by the computing module 130a or stored in the database module 140a with the at least one external device.

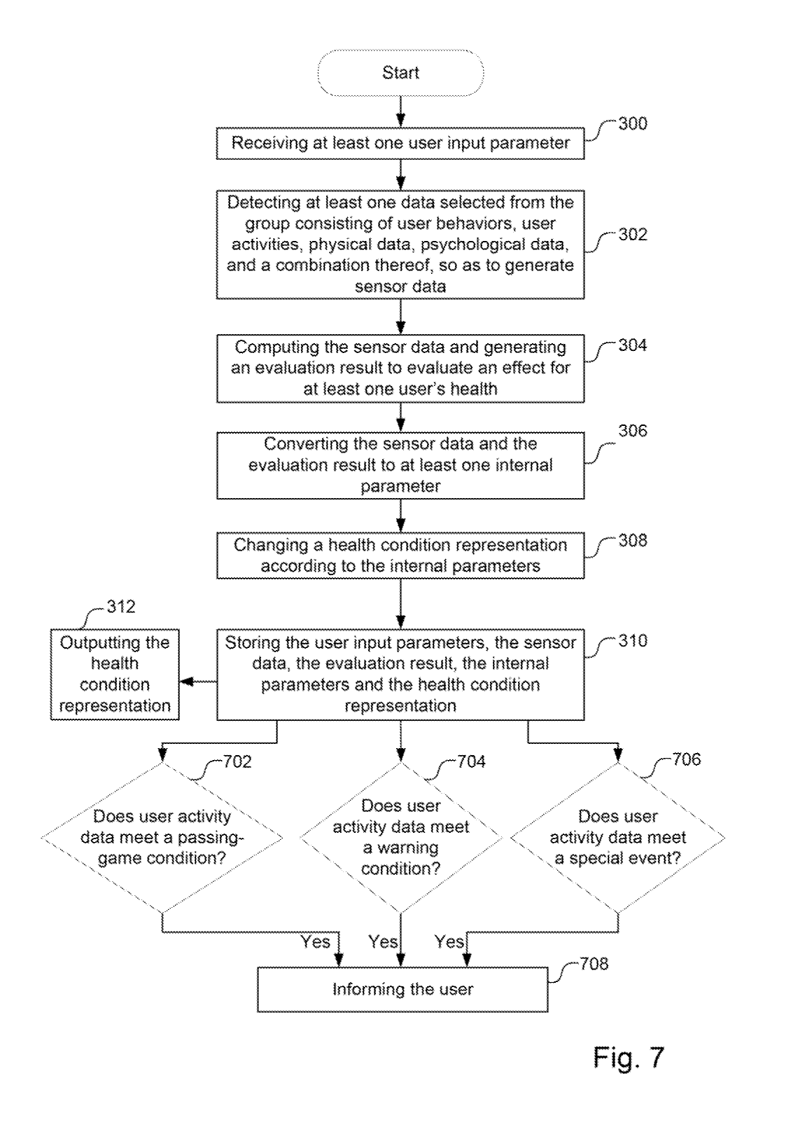

FIG. 7 is a flow chart of a health improvement method in accordance with one another embodiment of the present disclosure.

The health improvement method could be applied in the health improvement system 100 illustrated in FIG. 1 and is not limited herein. For convenience and clarity, the following descriptions about the health improvement method are illustrated based on the health improvement system 100 illustrated in FIG. 1.

Compared with the health improvement method illustrated in FIG. 3, the health improvement method illustrated in FIG. 7 further includes steps 702, 704, 706 and 708. In step 702, the computing module 130 determines whether the user activity data meets the passing-game condition according to the internal parameter. In step 704, the computing module 130 determines whether the user activity data meets the warning condition according to the internal parameter. In step 706, the computing module 130 determines whether the user activity data meets the special event according to the internal parameter. When the user activity data meets the passing-game condition, the warning condition or the special event, in step 708, the interaction module 110 informs the user.

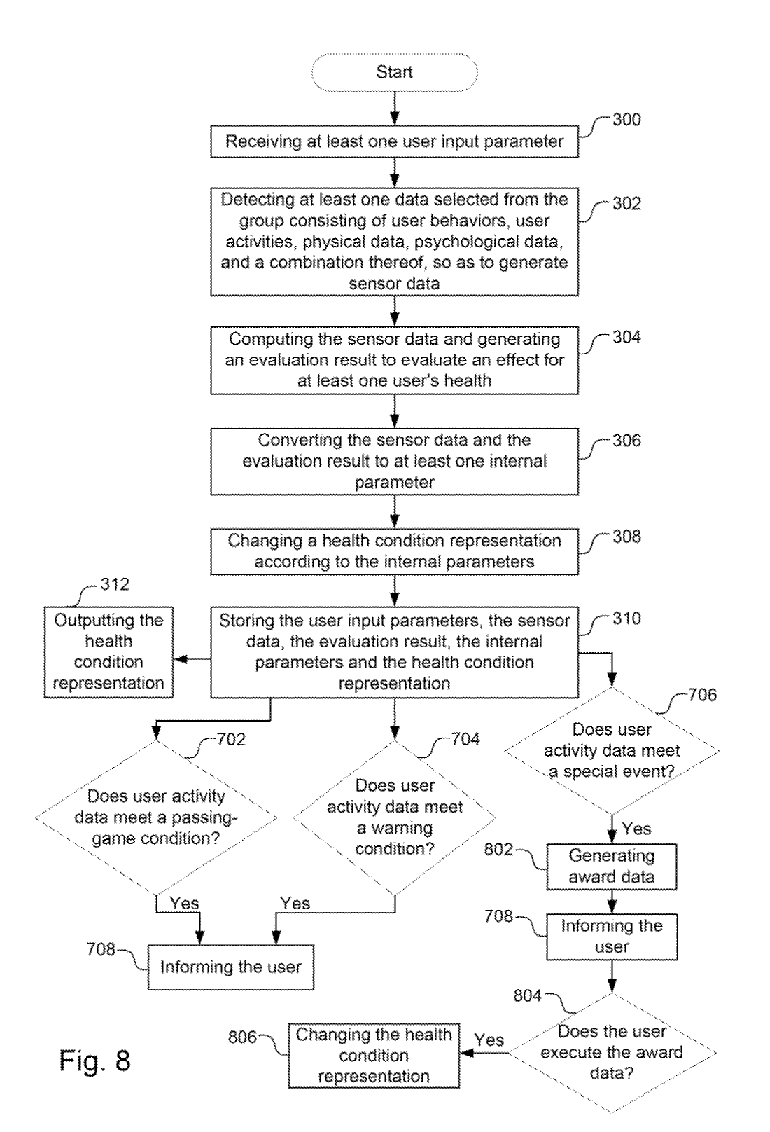

FIG. 8 is a flow chart of a health improvement method in accordance with one another embodiment of the present disclosure.

The health improvement method could be applied in the health improvement system 100 illustrated in FIG. 1 and is not limited herein. For convenience and clarity, the following descriptions about the health improvement method are illustrated based on the health improvement system 100 illustrated in FIG. 1.

Compared with the health improvement method illustrated in FIG. 7, the health improvement method illustrated in FIG. 8 further includes steps 802, 804 and 806. When the user activity data meets the special event, in step 802, the computing module 130 generates the award data. In step 804, the computing module 130 determines whether the user executes the award data according to the user input parameters 112. When the user executes the award data, in step 806, the computing module 130 changes the health condition representation 114.

From the above embodiments, by detecting the at least one data selected from the group consisting of user behaviors, user activities, user physical data, user psychological data, and a combination thereof, and converting the at least one data selected from the group consisting of user behaviors, user activities, user physical data, user psychological data, and a combination thereof to the changes of virtual characters correspondingly, the users could understand the effect for their health resulting from their behaviors, activities, physical data, psychological data, or a combination thereof. In this way, the users are encouraged to keep good habits and their health conditions are improved.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A health improvement system, comprising:
at least one sensor module configured for detecting at least one data selected from the group consisting of user behaviors, user activities, user physical data, user psychological data, and a combination thereof, so as to generate sensor data;
a computing module configured for computing the sensor data, for generating an evaluation result to evaluate an effect for at least one user's health, for converting the sensor data and the evaluation result to at least one internal parameter, and for changing a health condition representation according to the internal parameter;
an interaction module configured for receiving at least one user input parameter and for outputting the health condition representation; and
a database module configured for storing the user input parameters, the sensor data, the evaluation result, the internal parameters and the health condition representation.

2. The health improvement system of claim 1, wherein the computing module is further configured for searching and determining whether user data exists, wherein the user data is generated from the user input parameters or the sensor data; if the user data exists, the computing module is further configured for loading the user data and the health condition representation; and if the user data doesn't exist, the computing module is further configured for generating the user data and the health condition representation according to the user input parameters.

3. The health improvement system of claim 1, wherein the computing module is further configured for adjusting a difficulty level according to the user input parameters.

4. The health improvement system of claim 1, further comprising:
a network module configured for exchanging information generated by the computing module or stored in the database module with at least one external device.

5. The health improvement system of claim 4, wherein the external device comprises a second health improvement system, a social network, a server, or a combination thereof.

6. The health improvement system of claim 4, wherein the information comprises the health condition representation, achievement information, competition information or ranking information.

7. The health improvement system of claim 1, wherein the health condition representation comprises an appearance icon of a virtual character.

8. The health improvement system of claim 1, wherein the user input parameters comprise user age data, user gender data, user height data, user weight data, a parameter for choosing virtual characters, a parameter for adjusting difficulty level, a system order, a game order, or a combination thereof.

9. The health improvement system of claim 1, wherein the computing module is further configured for determining whether user activity data meets a passing-game condition, a warning condition or a special event; and when the user activity data meets the passing-game condition, the warning condition or the special event, the interaction module is further configured for informing the user.

10. The health improvement system of claim 9, wherein when the user activity data meets the special event, the computing module is further configured for generating award data, and for determining whether the user executes the award data; and when the user executes the award data, the computing module is further configured for changing the health condition representation.

11. A health improvement method, comprising:
receiving at least one user input parameter;
detecting at least one data selected from the group consisting of user behaviors, user activities, physical data, psychological data, and a combination thereof, so as to generate sensor data;
computing the sensor data and generating an evaluation result to evaluate an effect for at least one user's health;
converting the sensor data and the evaluation result to at least one internal parameter;
changing a health condition representation according to the internal parameter;
storing the user input parameters, the sensor data, the evaluation result, the internal parameters and the health condition representation; and
outputting the health condition representation.

12. The health improvement method of claim 11, further comprising:
searching and determining whether user data exists, wherein the user data is generated from the user input parameters or the sensor data;
if the user data existing, loading the user data and the health condition representation; and
if the user data not existing, generating the user data and the health condition representation according to the user input parameters.

13. The health improvement method of claim 11, further comprising:
adjusting a difficulty level according to the user input parameters.

14. The health improvement method of claim 11, further comprising:
exchanging information with at least one external device.

15. The health improvement method of claim 14, wherein the external device comprises a second health improvement system, a social network, a server, or a combination thereof.

16. The health improvement method of claim 14, wherein the information comprises the health condition representation, achievement information, competition information or ranking information.

17. The health improvement method of claim 11, wherein the health condition representation comprises an appearance icon of a virtual character.

18. The health improvement method of claim 11, wherein the user input parameters comprise user age data, user gender data, user height data, user weight data, a parameter for choosing virtual characters, a parameter for adjusting difficulty level, a system order, a game order, or a combination thereof.

19. The health improvement method of claim 11, further comprising:
determining whether user activity data meets a passing-game condition, a warning condition or a special event; and when the user activity data meeting the passing-game condition, the warning condition or the special event, informing the user.

20. The health improvement method of claim 19, further comprising:
when the user activity data meeting the special event, generating award data;
determining whether the user executes the award data; and
when the user executing the award data, changing the health condition representation.

21. A non-transitory computer-readable storage medium with a computer program to execute a health improvement method, wherein the health improvement method comprises:
receiving at least one user input parameter;
detecting at least one data selected from the group consisting of user behaviors, user activities, physical data, psychological data, and a combination thereof, so as to generate sensor data;
computing the sensor data and generating an evaluation result to evaluate an effect for at least one user's health;
converting the sensor data and the evaluation result to at least one internal parameter;
changing a health condition representation according to the internal parameter;
storing the user input parameters, the sensor data, the evaluation result, the internal parameters and the health condition representation; and
outputting the health condition representation.
